(19) United States
(12) Patent Application Publication
(10) Pub. No.: US 2002/0193342 A1
(43) Pub. Date: Dec. 19, 2002

(54) Modifying undesirable tastes

(76) Inventors: John P. Hamman, Baltimore, MD (US); Gary J. Calton, Elkridge, MD (US)

Correspondence Address: Gary J. Calton, 5331 Landing Road, Elkridge, MD, 21075, US

(21) Appl. No.: 09/852,182

(22) Filed: May 9, 2001

(51) Int. Cl. A61K 31/715; A61K 38/00

(52) U.S. Cl. 514/53; 424/439; 514/400; 514/419; 514/2

(57) ABSTRACT

Masking compositions are provided comprising at least one unpleasant tasting amino acid other than arginine, protein hydrolysate and/or protein component wherein the taste of the unpleasant tasting component is masked with the addition of sucralose to provide organoleptically acceptable compositions. Masking compositions in accordance with the invention may then be incorporated in beverages, foods, bars, or candies to provide nutrient products.

### TECHNICAL FIELD

The present invention relates to the use of sucralose as an agent for reducing or inhibiting the unpleasant tastes of amino acids and proteins. In particular, this invention relates to organoleptically acceptable amino acid, protein hydolysate and protein compositions containing sucralose and to methods for the preparation and use of said compositions.

### BACKGROUND OF THE INVENTION

Consumers do not care for the bitter and metallic tastes of amino acids, protein hydrolysates and proteins in the broadest sense. Typical doses of amino acids, protein hydrolysates and proteins are sufficiently large that one would need to take a large number of pills or a large volume of powder to achieve the desired therapeutic dosage level. The desire for improved palatability of oral compositions having unpleasant tasting amino acid and protein components has prompted the development of numerous formulations and methods of removing undesirable tastes in orally administrable compositions.

Compounds conventionally used to mask bitter flavors in oral compositions have included, inter alia, phosphorylated amino acids (U.S. Pat. No. 5,766,622); acidic amino acids (U.S. Pat. No. 4,517,379); chitosan (Japanese Patent Application No. 04-009,335); cyclodextrins (U.S. Pat. No. 5,024,997); liposomes (U.S. Pat. No. 5,009,819); lecithin or lecithin like substances (Japanese Patent Application No. 62-265,234); surfactants (U.S. Pat. No. 5,439,671); salts (U.S. Pat. No. 5,262,179); and the like.

Attempts to mask unpleasant tastes in oral compositions have also included such techniques as coating or microencapsulation (European Patent Application No. 551,820); functional group alteration (U.S. Pat. No. 5,350,839); and structural matrix forms of taste masking have been used. Oral compositions employing such technology have incorporated agents such as silicate clays (U.S. Pat. Nos. 3,140,978 and 4,581,232); acrylic acid copolymers (U.S. Pat. No. 5,286,489); gums (U.S. Pat. No. 5,288,500); cellulose (U.S. Pat. No. 5,192,563); and waxes in an effort to provide improved tasting compositions.

Certain intense sweeteners have been used to offset the associated bitter aftertaste or unpleasant offnote of other intense sweeteners. For example, United Kingdom patent application no. 2154850A discloses the use of a combination of at least two intense sweeteners to modify the associated unpleasant taste of one of the sweeteners (cyclamate). The combination of the two sweeteners is said to provide a preferred sweetness. Specifically, a composition is disclosed for sweetening a beverage such as a cola, tea or coffee which comprises combining a chlorosucrose sweetener with a cyclamate, which is used either alone or is in combination with other sweeteners.

U.S. Pat. No. 4,495,170, discloses synergistic sweetening compositions which comprise a mixture of a chlorodeoxysugar and another sweetening agent which has an associated bitter taste. The chlorodeoxysugars are selected from the group consisting of chlorodeoxysucroses and chlorodeoxygalactosucroses. The bitter tasting sweetening agent is selected from the group consisting of Saccharin, stevioside and Acesulfame-K.

U.S. Pat. No. 4,535,396, teaches a method of masking the bitter taste and enhancing the sweet taste of Acesulfame-K by combining the bitter-tasting intense sweetener with the sweetener Alitame.

U.S. Pat. No. 4,158,068 discloses a sweetener mixture to improve the saccharose-like quality of acetosulfame-K. Specifically, acetosulfame-K is combined with at least one intense sweetener selected from the group consisting of aspartyl peptide ester sweeteners, sulfamate sweeteners, sulfimide sweeteners and dihydrochalcone sweeteners.

U.S. Pat. No. 5,013,716 discloses unpleasant taste masking compositions for medicament drugs or chewing gum flavors having a bitter taste or unpleasant off-note with a chlorodeoxysugar to nullify the taste or unpleasant off-note of the medicament drug. Medicaments are said to include dietary supplements, including vitamins and minerals, such as niacin, pantothenic acid, vitamin B6, thiamine hydrochloride, riboflavin, potassium iodide, potassium chloride, cupric sulfate and ferrous sulfate. Although amino acids and proteins are sometimes described as nutritional supplements, the description of dietary supplements of U.S. Pat. No. 5,013,716 does not mention this grouping of bitter, metallic, fishy and offtasting dietary supplements materials, thus, no conclusion could be drawn about the efficacy of a chlorodeoxysugar to mask the unpleasant taste of the amino acids or proteins that taste bad.

U.S. Pat. No. 6,143,786 discloses compositions for controlling diabetes mellitus consisting of arginine, an organic acid, phosphoric acid and a high intensity sweetener.

Recently, it has been suggested that certain amino acids, when used as supplements to a normal diet in relatively large doses, have salutary effects on kidney health (Walser et al., “Can renal replacement be deferred by a supplemented very low protein diet?” J. Am. Soc. Nephrol., Vol. 10(1), (January 1999), pages 110-6; and weight control (Geliebter et al., “Oral L-histidine fails to reduce taste and smell acuity but induces anorexia and urinary zinc excretion”, Am. J. Clin. Nutr., Vol. 34(1), (January 1981), pages 119-20).

In particular, arginine has also been shown to possess a number of beneficial effects (A. Barbul, Amino Acid Metabolism & Therapy in Health & Nutritional Disease, CRC Press, Inc., Boca Raton, Fla., 1995, chapter 25, pp 361-372) including wound healing and improved immune function. Arginine has also been shown to lower cholesterol in humans (U.S. Pat. No. 5,157,022), to prevent atherosclerosis (U.S. Pat. Nos. 5,945,452 and 5,428,070) and to enhance athletic performance (U.S. Pat. No. 6,117,872).

Arginine has an unpleasant fishy taste and aftertaste. The art of masking the unpleasant taste of arginine has been the subject of intense investigation (see for instance U.S. Pat. No. 6,063,432 which discloses a health bar of which the major portion consists of fruit pastes in order to provide a palatable form of arginine).

Accordingly, there exists a need to develop formulations for amino acids, protein hydrolysates, and proteins which are organoleptically acceptable.

### SUMMARY OF THE INVENTION

It has now been found that sucralose is an effective agent to mask the unpleasant tastes and off-flavor of amino acids, protein hydrolysates and proteins or analogs thereof, excluding arginine. Accordingly, the present invention pertains to compositions which contain sucralose as a component to mask the bitterness or off-note of amino acids, protein hydrolysates and proteins. The present invention provides taste masking compositions which have an improved taste without an unpleasant, bitter/metallic taste or aftertaste, as well as ingestible products which contain the unpleasant taste masking compositions. The amount of sucralose added is above that normally needed to give a sweet taste to a composition having no bitter component such as amino acids, protein hydrolysates and proteins that are bad tasting.

It is therefore an advantage of the present invention to provide a method for inhibiting the undesirable taste of oral compositions, e.g. food, drinks, dietary supplement and other pharmaceuticals, which contains amino acids, protein hydrolysates and protein components.

It is also an advantage of the present invention to provide pleasant tasting formulations of amino acids, protein hydrolysates and proteins in pharmaceutically acceptable dosages in a bio-available and organoleptically acceptable form in humans.

Other aspects and advantages of the present invention will become apparent from the following description, taken in conjunction with the ensuing examples and claims.

### DETAILED DESCRIPTION OF THE INVENTION

In accordance with the present invention, oral compositions are provided which are useful to mask the undesirable taste of amino acid, protein hydrolysate or protein components.

For purposes of this invention, the term “sucralose” as used herein, is defined as 1,6-dichloro-1,6-dideoxy-β-D-fructofuranosyl-4-chloro-4-deoxy-α-D-galactopyranoside.

The term “oral compositions” as used herein, is defined as any product which in the ordinary course of usage is intentionally swallowed or ingested by humans, such as foods, drinks, pharmaceuticals, dietary supplements and the like.

The phrase “mask” as used herein, is defined as covering, disguising, and/or obscuring the taste of an amino acid, protein hydrolysate or protein component by the addition of a compound, wherein the amino acid, protein hydrolysate or protein component remains unchanged, but its bad taste is not perceived by a human consuming said composition.

In one aspect of the invention, the compositions comprise at least one undesirable tasting amino acid, protein hydrolysate or protein component, and an amount of sucralose sufficient to inhibit or substantially inhibit the taste of said undesirable tasting component. In general, the amount of sucralose useful to prepare compositions in accordance with the present invention is from about 0.001% to 15% by weight of sucralose, preferably, from about 0.1% by weight to about 12% by weight of sucralose, and most preferably, from about 0.5% by weight to about 10% by weight of sucralose where said weights refer to the dry weights of the components.

The amino acids useful to prepare compositions in accordance with the invention include, but are not limited to, one or more amino acids and their analogs selected from the group consisting of L-alanine, L-aspartic acid, L-citrulline, L-cystine, L-glutamic acid, L-glutamine, L-histidine, L-isoleucine, L-leucine, L-lysine, L-methionine, D,L-methionine L-omithine, L-phenylalanine, L-proline, L-serine, L-threonine, L-tryptophan, L-tyrosine, L-valine, the keto analogs of the previously listed amino acids and the hydroxy analogs of the previously listed amino acids, creatine, carnitine and mixtures thereof. The keto and hydroxy analogs of the amino acids have the amine group replaced with a keto or hydroxyl group, respectively. A more desirable group consists of one or more amino acids or their analogs selected from the group consisting of L-glutamine, L-histidine, L-isoleucine, L-leucine, L-lysine, L-methionine, L-phenylalanine, L-threonine, L-tryptophan, L-tyrosine, and L-valine. Yet another desirable group of the amino acids consists of the branched chain amino acids, L-leucine, L-isoleucine and L-valine.

Typically, compositions in accordance with this aspect of the invention will comprise amino acids in the amount ranging from about 0.01% to 99.99% by dry weight of the total composition, preferably, from about 0.5% to about 99.5% by dry weight of the total composition of the total composition.

The amino acids may be in the form of protein hydrolysates or mixtures of protein hydrolysates, e.g., hydrolyzed egg albumin or hydrolyzed whey, either fully or partially hydrolyzed.

Proteins may also be included as a component in the oral compositions of the invention. Suitable proteins include, but are not limited to, those obtained from soy, whey, casein, wheat, corn, and albumin. Typically, proteins are incorporated into the composition in amounts ranging from about 0.01 to about 99.99% by weight.

Compositions in accordance with the present invention may also include as a supplement at least one carbohydrate (saccharide), often free of glucose as the monomer or oligomer, e.g. sucrose. The total amount of saccharides, normally hexasaccharides will be in the range of about 0.01 to about 90 weight %, usually in the range of about 1 to about 50 weight %. Saccharides of particular interest include sucrose, maltitol, conveniently used as a syrup, fructose, conveniently used as a solid, honey, rice syrup, corn syrup, high fructose corn syrup, high maltose corn syrup, and the like. Maltitol, when present, will generally be present in from about 2 to about 50 weight %. Fructose, when present will be in the range of about 2 to about 50 weight %. Mannitol may be substituted in whole or part for the other saccharides, particularly reduced saccharine.

Compositions in accordance with the present invention may also contain minor ingredients such as, for example, lipids, fiber for reduction of cholesterol, e.g. oat fiber, vegetable powder, etc., colorants, e.g. beet powder, annatto, carmine, caramel color, FD&C colors, etc., flavoring, e.g. chocolate, fruit, vanilla, confectionary particles, almonds etc., other artificial sweeteners, e.g. acesulfam k, aspartame, alitame, stevioside, etc., and anti-staling agents such as surfactants.

Generally, such minor ingredients are present in the formulation in an amount ranging in total from about 2 to 20 weight % and individually from about 0.5 to 7.5, preferably about 0.5 to 5.0, weight %, to provide flavor, texture and/or appearance. Where the minor ingredient are lipids, they will preferably be present in less than 10% by weight, and desirably will be primarily polyunsaturated, including omega-3 polyunsaturated lipids. Where the minor ingredient is fiber, it will preferably be present in a range of about 0 to about 20% by weight. Where the minor ingredient is flavoring, it will preferably be present in a range of about 0.001% to about 15% by weight.

In addition to the ingredients described hereinabove, other functional ingredients may be added to enhance flavor texture, appearance and as a processing aid. Such ingredients are usually present in amounts not to exceed 3 weight %, preferably not 2 weight %, of the total formulation.

Oral compositions of the present invention may be used in many distinct physical forms well known in the art. Without being limited thereto, such physical forms include free forms, such as beverages, spray dried, powdered, beaded, and encapsulated forms, and mixtures thereof as well as in formulations of candies, gums, bars and the like.

Typically, compositions in accordance with the present invention are prepared by thoroughly mixing the amino acid, protein and/or protein hydrolysate components with sucralose using mixing techniques common to the baking, beverage and chemical industries such as mixers, blenders or extruders. Other materials capable of imparting such characteristics as texture or flavor, e.g. other sweeteners, flavors, colorants, water, lipids, thickeners, emulsifiers, organics, gums, and the like, may also be blended into the mixture. The mixture is thereafter packaged in any desirable form.

The following examples illustrate the practice of the present invention, but are not intended to limit its scope.

### EXAMPLES

### Example 1

Arginine free base, which has a bad aftertaste characterized as “fishy” or arginine hydrochloride, which has a “sour” or “metallic” aftertaste, was mixed with sugar or a high intensity sweetener including, aspartame, saccharin, sucralose or as a control, sugar, in water. The mixtures were then tasted by a trained taste panel and evaluated for retention of the bitter/metallic or bad aftertaste and compared to an unsweetened mixture. All member of the taste panel could taste the bitter aftertaste of saccharin. Results of the taste test are recorded in Table 1 below.

TABLE 1

| | | | | dry | | wet | |
|---|---|---|---|---|---|---|---|
| | Amino | Sweet- | (S) | wt | | wt | |
| | acid wt | ener | Wt | % | Water | % | |
| Amino Acid | (g) | (S) | (mg) | (S) | (g) | (S) | Taste |
| Arginine | 1.75 | | 0 | 0 | 110 | 0 | Fishy |
| Arginine | 1.75 | Su- | 170 | 9 | 110 | 0.2 | sweet- |
| | | cralose | | | | | fishy |
| Arginine | 1.75 | Aspar- | 170 | 9 | 110 | 0.2 | sweet- |
| | | tame | | | | | fishy |
| Arginine | 1.75 | Aspar- | 680 | 28 | 110 | 0.7 | sweet- |
| | | tame | | | | | fishy |
| Arginine | 1.75 | Saccha- | 346 | 16 | 110 | 0.3 | sweet- |
| | | rin | | | | | fishy |
| | | | | | | | metallic |
| Arginine | 1.75 | Sugar | 1730 | 91 | 110 | 15 | sweet- |
| | | | 0 | | | | fishy |
| Arginine.HCl | 1.75 | | 0 | 0 | 110 | 0 | metallic |
| Arginine.HCl | 1.75 | Aspar- | 170 | 9 | 110 | 0.2 | sweet- |
| | | tame | | | | | metallic |
| Arginine.HCl | 1.75 | Su- | 170 | 9 | 110 | 0.2 | sweet- |
| | | cralose | | | | | metallic |

None of the sweeteners were effective in reducing the bad taste or aftertaste of arginine when mixed with either the free base or as its hydrochloride salt.

### Example 2

A mixture of amino acids consisting of L-histidine, 7.97%; L-isoleucine, 10.14%; L-leucine, 15.94%; L-lysine, 11.59%; L-methionine, 15.94%; L-phenylalanine, 15.94%; L-threonine, 7.25%; L-tryptophan, 3.62%; and L-valine, (10.5 g), which give a bitter/metallic aftertaste when ingested orally, was mixed with sucralose (1.02 g) (8.8% sucralose content). 1.0 g of the mixture was placed in 40 mL of water. The result was a sweet tasting formulation with no bitter or metallic aftertaste. A control solution having no sucralose was bitter with a long lasting metallic aftertaste.

### Example 3

A mixture of amino acids consisting of L-histidine, 7.97%; L-isoleucine, 10.14%; L-leucine, 15.94%; L-lysine, 11.59%; L-methionine, 15.94%; L-phenylalanine, 15.94%; L-threonine, 7.25%; L-tryptophan, 3.62%; and L-valine, (147 g), which give a bitter/metallic aftertaste when ingested orally, was mixed with sucralose (6.02 g) (3.9% sweetener concentration). One g of the mixture was placed in 40 mL of water (0.01% sweetener). The result was a sweet tasting formulation with no bitter or metallic aftertaste.

### Example 4

A commercially prepared egg albumin hydrolysate 1 g, characterized as bitter in taste, was mixed with sucralose, 0.02 mL of a 25% solution, in 25 mL of water and the taste was evaluated. A sweet smooth formulation was obtained. When the experiment was duplicated with 20 g of sugar rather than the sucralose, a bitter off-note was present. When the experiment was duplicated with 0.03 g of aspartame rather than the sucralose, a bitter off-note was present. Only sucralose masked the bitter aftertaste of the protein hydrolysate.

All publications and patent applications cited in this specification are herein incorporated by reference.

Although the foregoing invention has been described in some details by way of illustration and examples for purposes of clarity of understanding, it will be readily apparent to those of ordinary skill in the art in light of the teachings of this invention that certain changes and modifications may be made thereto without departing from the spirit or scope of the appended claims.

We claim:

1. A taste masking composition comprising at least one amino acid component having an unpleasant taste and sucralose in an amount sufficient to mask the unpleasant taste of the at least one amino acid component, wherein said at least one amino acid is not arginine.

2. The composition of claim 1 wherein sucralose is present in an amount ranging from about 0.001 to about 15 dry weight % of the composition.

3. The composition of claim 2 wherein sucralose is present in an amount ranging from about 0.1 to about 12 dry weight % of the composition.

4. The composition of claim 3 wherein sucralose is present in an amount ranging from about 0.5 to about 10 dry weight % of the composition.

5. The composition of claim 1 wherein said at least one amino acid is selected from the group consisting of L-alanine, L-aspartic acid, L-citrulline, L-cystine, L-glutamic acid, L-glutamine, L-histidine, L-isoleucine, L-leucine, L-lysine, L-methionine, D,L-methionine, L-omithine, L-phenylalanine, L-proline, L-serine, L-threonine, L-tryptophan, L-tyrosine, L-valine, creatine, carnitine and mixtures thereof.

6. The composition of claim 1 wherein said at least one amino acid component comprising an amino acid analog selected from the group consisting of the hydroxy analogs and keto analogs of the amino acids.

7. The composition of claim 5 wherein said amino acid consists of a mixture of L-leucine, L-isoleucine and L-valine.

8. The composition of claim 5 wherein said amino acid is L-histidine.

9. The composition of claim 5, wherein said amino acid is a mixture of amino acids selected from the group comprising L-histidine, L-isoleucine, L-leucine, L-lysine, L-methionine, L-phenylalanine, L-threonine, L-tryptophan, L-tyrosine and L-valine.

10. The composition of claim 9 wherein said amino acid consists of a mixture of L-leucine, L-isoleucine and L-valine.

11. A taste masking composition comprising at least one protein hydrolysate component having an unpleasant taste and sucralose in an amount sufficient to mask the unpleasant taste of the at least one protein hydrolysate component.

12. The composition of claim 11 wherein sucralose is present in an amount of from about 0.001 to about 15 dry weight % of the composition.

13. The composition of claim 12 wherein sucralose is present in an amount ranging from about 0.1 to about 12 dry weight % of the composition.

14. The composition of claim 13 wherein sucralose is present in a amount ranging from about 0.5 to about 10 dry weight % of the composition.

15. A taste masking composition comprising at least one protein component having an unpleasant taste and sucralose in an amount sufficient to mask the unpleasant taste of the at least one protein component.

16. The composition of claim 15 wherein sucralose is present in an amount ranging from about 0.001 to about 15 dry weight % of the composition.

17. The composition of claim 16 wherein sucralose is present in an amount ranging from about 0.1 to about 12 dry weight % of the composition.

18. The composition of claim 17 wherein sucralose is present in an amount ranging from about 0.5 to about 10 dry weight % of the composition.

19. A method of masking the unpleasant taste of an amino acid component in an amino acid containing composition, said method comprising adding to a composition containing at least one unpleasant tasting amino acid sucralose in an amount sufficient to mask the unpleasant taste of the at least one amino acid, wherein said at least one amino acid component is not arginine.

20. The method of claim 19 wherein sucralose is added in an amount ranging from about 0.001 to about 15 dry weight % of the composition.

21. The method of claim 20 wherein sucralose is added in an amount ranging from about 0.1 to about 12 dry weight % of the composition.

22. The method of claim 21 wherein sucralose is added in an amount ranging from about 0.5 to about 10 dry weight % of the composition.

23. The method of claim 19 wherein said at least one amino acid is selected from the group consisting of L-alanine, L-aspartic acid, L-citrulline, L-cystine, L-glutamic acid, L-glutamine, L-histidine, L-isoleucine, L-leucine, L-lysine, L-methionine, D,L-methionine, L-omithine, L-phenylalanine, L-proline, L-serine, L-threonine, L-tryptophan, L-tyrosine, L-valine, creatine, carnitine and mixtures thereof.

24. The method of claim 19 wherein said at least one amino acid comprises an amino acid analog selected from the group consisting of the hydroxy analogs and keto analogs of the amino acids.

25. The method of claim 23 wherein said amino acid consists of a mixture of L-leucine, L-isoleucine and L-valine.

26. The method of claim 23 wherein said amino acid is L-histidine.

27. The method of claim 23 wherein said amino acid is a mixture of amino acids selected from the group consisting of L-histidine, L-isoleucine, L-leucine, L-lysine, L-methionine, L-phenylalanine, L-threonine, L-tryptophan, L-tyrosine and L-valine.

28. The method of claim 27 wherein said amino acid consists of a mixture of L-leucine, L-isoleucine and L-valine.

29. A method of masking the unpleasant taste of a protein component in a protein containing composition, said method comprising adding to a composition containing at least one unpleasant tasting protein sucralose in an amount sufficient to mask the unpleasant taste of the at least one protein.

30. The method of claim 29 wherein sucralose is added in an amount of from about 0.001 to about 15 dry weight % of the composition.

31. The method of claim 30 wherein sucralose is added in an amount ranging from about 0.1 to about 12 dry weight % of the composition.

32. The method of claim 31 wherein sucralose is added in a amount ranging from about 0.5 to about 10 dry weight % of the composition.

33. A method of masking the unpleasant taste of a protein hydrolysate component in a protein hydrolysate containing composition, said method comprising adding to a composition containing at least one unpleasant tasting protein hydrolysate sucralose in an amount sufficient to mask the unpleasant taste of the at least one protein.

34. The method of claim 33 wherein sucralose is added in an amount of from about 0.001 to about 15 dry weight % of the composition.

35. The method of claim 34 wherein sucralose is added in an amount ranging from about 0.1 to about 12 dry weight % of the composition.

36. The method of claim 35 wherein sucralose is present in a amount ranging from about 0.5 to about 10 dry weight % of the composition.
